(19) United States
(12) Patent Application Publication
(10) Pub. No.: US 2015/0150515 A1
(43) Pub. Date: Jun. 4, 2015

(54) RESPIRATION RATE EXTRACTION FROM CARDIAC SIGNALS

(71) Applicant: OBS MEDICAL LIMITED, Oxfordshire (GB)

(72) Inventor: Iain Guy David Strachan, Oxfordshire (GB)

(21) Appl. No.: 14/404,349

(22) PCT Filed: May 28, 2013
(86) PCT No.: PCT/GB2013/051406

§ 371 Date: Nov. 26, 2014

(30) Foreign Application Priority Data
May 28, 2012 (GB) 1209413.2

(51) Int. Cl. A61B 5/00; A61B 5/0402; A61B 5/0205

(52) U.S. Cl. CPC A61B 5/7278; A61B 5/0205; A61B 5/7257; A61B 5/0402; A61B 5/0816

(57) ABSTRACT

At least two waveforms indicative of respiration are extracted from cardiac signals such as PPG or ECG. The extracted waveforms are converted into the frequency domain and multiplied together to form a combined frequency response function representing the frequency content of the combined respiratory waveform. Up to three peaks in the combined frequency response function are identified and averaged to provide an improved estimate of the breathing rate.

The present invention relates to a method of and apparatus for obtaining an improved estimate of respiration rate from cardiac signals from a human or animal subject.

It is well known that cardiac signals such as electrocardiograms (ECG) or photoplethysmographs (PPG) contain detectable respiration information via three different physiological mechanisms, namely:

(i) Respiratory Sinus Arrhythmia—the regular variation of the heart rate during the respiration cycle which can be observed by measuring the beat-to-beat times between corresponding reference points on the cardiac signal waveform (e.g. the R-peak of the ECG or maximum values of the PPG signal). During normal respiration the beat to beat times periodically increase and decrease as the subject breathes and this effectively constitutes a frequency modulation in the cardiac signal.

(ii) Waveform Amplitude Variability—in both PPG and ECG signals the height above a baseline of the various peaks in the signal periodically varies with breathing. This constitutes an amplitude modulation in the cardiac signal.

(iii) Baseline Variability—the PPG signal, in particular, includes a baseline level which varies as the subject breathes. Some types of pulse oximeter pre-process the PPG signal to remove this baseline, but where the raw signal is available, this periodic variation in the baseline is a measure of respiration.

Both ECG and PPG signals are capable of providing at least two signals which Include respiration information and thus form potential sources for a measurement of respiration rate. There are several motivations driving interest in these cardiac-derived measurements of respiration rate. Although it is possible to measure respiration more directly, for example by thoracic impedance pneumography or by an acoustic sensor on the windpipe, such sensors can be uncomfortable for a patient and can be subject to interference and signal loss. If respiration information can be derived from other sensors on the patient, such as ECG or PPG, it is more convenient for the patient. Also in post-processing of existing data a direct respiration rate measurement may not have been recorded whereas PPG and ECG often is. Consequently the ability accurately to derive the respiration rate from the existing cardiac data is useful.

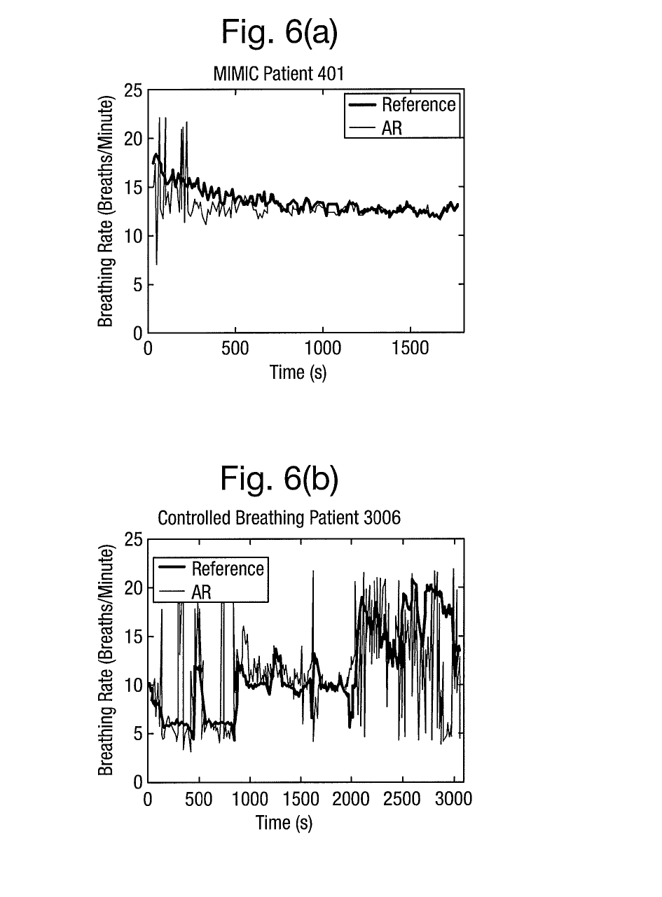

The processing of cardiac signals to derive respiration rate has been disclosed in, for example, EP-A-1463447, EP-A-2173242 and WO 2009/016334 A1. FIG. 6 of the accompanying drawings illustrates results obtained by the method of EP 2173242. In this method the PPG signal is processed by autoregression (AR) modelling to derive the complex domain poles of the frequency response function defined by the AR coefficients. In the complex domain the angle of a pole with respect to the real axis represents a frequency component present in the signal. Typically, with a tenth order AR model there will be five pairs of poles in complex conjugate pairs, each representing a frequency present in the signal, and one of these is assumed to be the breathing frequency. There has been considerable research in to how to pick the “correct” pole, to get the best breathing rate estimate and the simplest method is to pick the pole that is closest to the unit circle (which should give the sharpest resonance in the frequency response). However as can be seen from FIG. 6 although there is correlation between the breathing rate estimate produced by the AR model and a gold standard reference, there are also areas of significant disagreement. This can arise because the pole closest to the unit circle does not necessarily correspond to the breathing frequency, particularly if the signal is corrupted by large movement artefact. While techniques such as Kalman filtering can be used to improve the estimate, it is still possible for this technique to lock on to the incorrect pole and thus given an incorrect estimate of breathing rate.

The present invention is based on a spectral analysis of a cardiac-derived respiration signal, but instead of selection of the breathing rate by choice of a single pole from an AR model, the selection is based on the shape of the continuous frequency response curve obtained by converting the cardiac-derived signal into the frequency domain. In particular it focuses on peaks in the frequency domain, which has the advantage that it does not necessarily lock on to the single highest peak. For example it is sometimes the case that in an AR model two lower magnitude poles that are positioned close together can give rise to a large peak in the frequency response. Consequently focussing on the peaks in the frequency response can lead to a better estimate of the true breathing rate.

Furthermore, with the present invention data fusion of the two different cardiac-derived respiration signals is performed by multiplying together the frequency response curves obtained from the two different cardiac-derived respiration signals and the spectral peaks in the resulting product are observed. This data fusion means that the true respiration signal is enhanced by the multiplication whereas artefactual peaks which are present in one signal but not the other are suppressed.

In more detail, therefore, one aspect of the present invention provides a method of obtaining an improved estimate of respiration rate from two cardiac-derived signals representative of respiration of a human or animal subject, comprising the steps of:

converting the two cardiac-derived signals into the frequency domain to generate two respective frequency response signals representing the spectral content of the cardiac-derived signals;

point-by-point multiplying together the frequency response signals and identifying the improved estimate of respiration rate from one or more spectral peaks in the product function resulting from the multiplication.

There may be one clear spectral peak in the product function, or there may be more than one. Preferably the proportion of the total energy corresponding to the peak or peaks is calculated, for example by computing the value of the area under the curve in a narrow range either side of the or each peak in a plot of the product function. If there is a single peak which has more than a predetermined proportion of the total energy, for example more than 40%, or more preferably more than 50%, it is regarded as representing the improved estimate of respiration rate.

Another aspect of the present invention provides a computer program comprising computer-executable code that when executed on a computer system causes the computer system to perform a method as defined above. A further aspect of the invention provides a computer-readable medium storing a computer program according to the preceding aspect of the invention.

A yet further aspect of the invention provides an apparatus for obtaining an improved estimate of respiration rate from two or more cardiac-derived signals representative of respiration, comprising:
a conversion section configured to convert the cardiac-derived signals into the frequency domain to generate respective frequency response signals representing the spectral content of the cardiac-derived signals; and
an analysis section configured to point-by-point multiply together the frequency response signals and configured to identify the improved estimate of respiration rate from one or more spectral peaks in the product function resulting from the multiplication.

The present invention allows for multiple peaks to be present in the spectrum. This can occur if, for example, the breathing rate is not uniform during the measurement period. In this case the improved estimate of respiration rate is preferably obtained by a weighted combination based on the relative heights of the multiple peaks. Preferably up to three peaks are considered on condition that the three peaks together have more than a predetermined proportion of the total energy in the signal, for example more than 40% or more preferably more than 50% as above.

In the event that it is not possible to identify three or fewer peaks having more than the predetermined proportion of the total energy, it is assumed that it is not possible to obtain the respiration rate estimate from the signal.

When computing the area under the product function to derive the energy associated with a peak preferably the computation is over a range of frequency corresponding to respiration rates of plus or minus 1.5 breaths per minute. Different ranges may be used if appropriate.

Although the invention requires a minimum of two cardiac-derived respiration rate signals, there may be more than two. Thus the invention is applicable to at least two cardiac derived respiration rate signals selected from the frequency and amplitude variations in each of the PPG and ECG signals, and also the baseline variability in the PPG signal. The two signals may be both obtained from the PPG or both from the ECG, or one or more from each of them.

To improve the estimate further it is possible to pre-process the cardiac-derived signals before converting them into the frequency domain. For example the cardiac derived signals can be narrow band filtered around the expected respiration rate (or a harmonic thereof).

It is also known to analyse an ECG signal by use of a Hidden Markov Model (HMM), in particular to segment the ECG signal into its different cardiac features. HMMs provide a confidence measure and if such a confidence measure is obtained it is possible to remove from the signal sections for which the confidence measure is lower than a predetermined threshold.

It is also possible to use the measured proportion of the total energy represented by the one or more spectral peaks in the product function as a measure of the quality of the estimate of respiration rate. The more signal energy present in the one or more spectral peaks corresponding to the estimate, the higher the confidence in the estimate.

Embodiments of the present invention will be further described by way of example with reference to the accompanying drawings in which:-

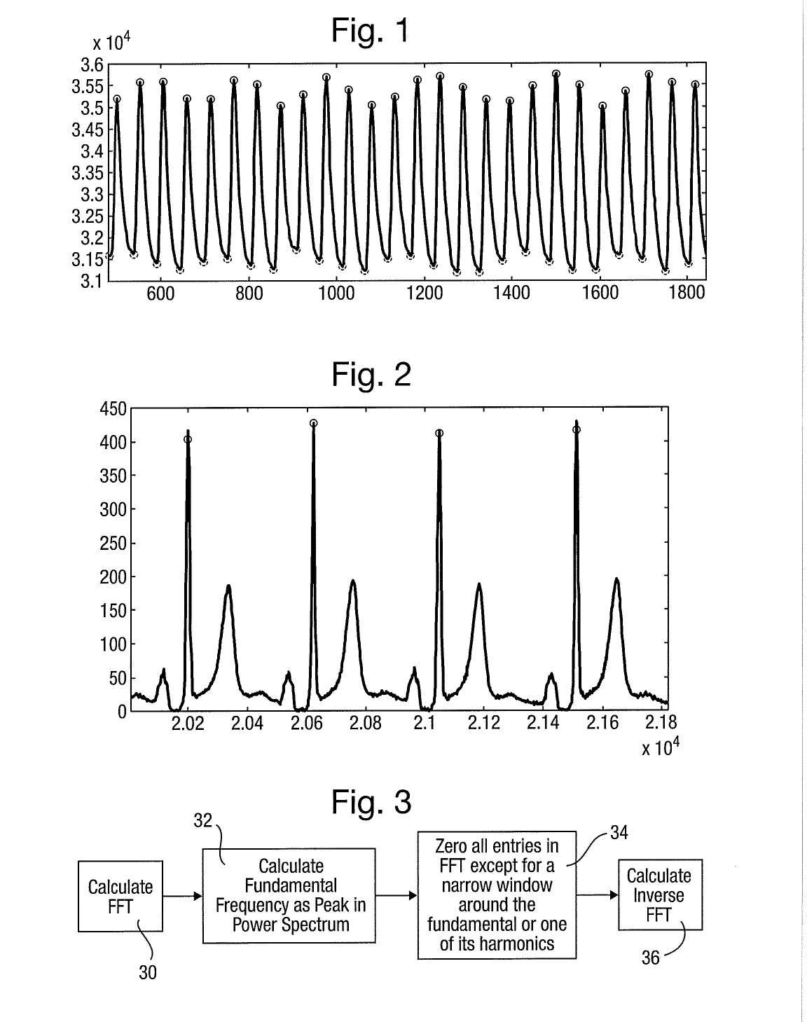

FIG. 1 illustrates peak detection on a PPG signal;

FIG. 2 illustrates peak detection on an ECG signal;

FIG. 3 illustrates schematically narrow band filtering of a signal;

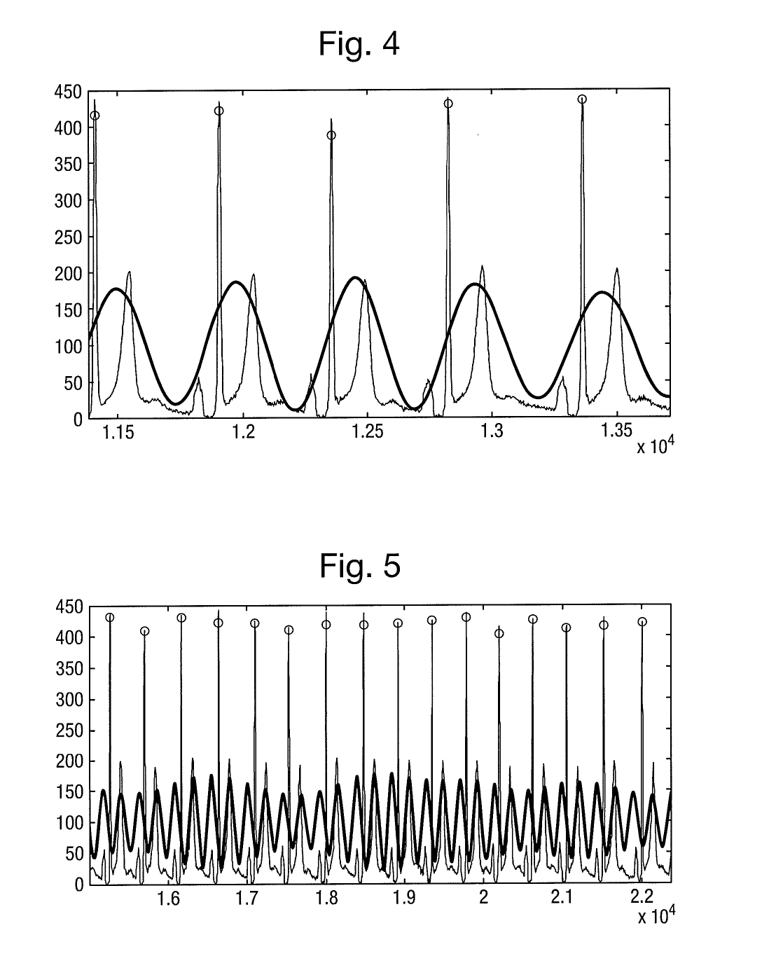

FIG. 4 illustrates an input and output from the process of FIG. 3;

FIG. 5 illustrates an alternative input and output for the process of FIG. 3;

FIG. 6(a) and (b) illustrate results of breathing rate detection in a prior art method;

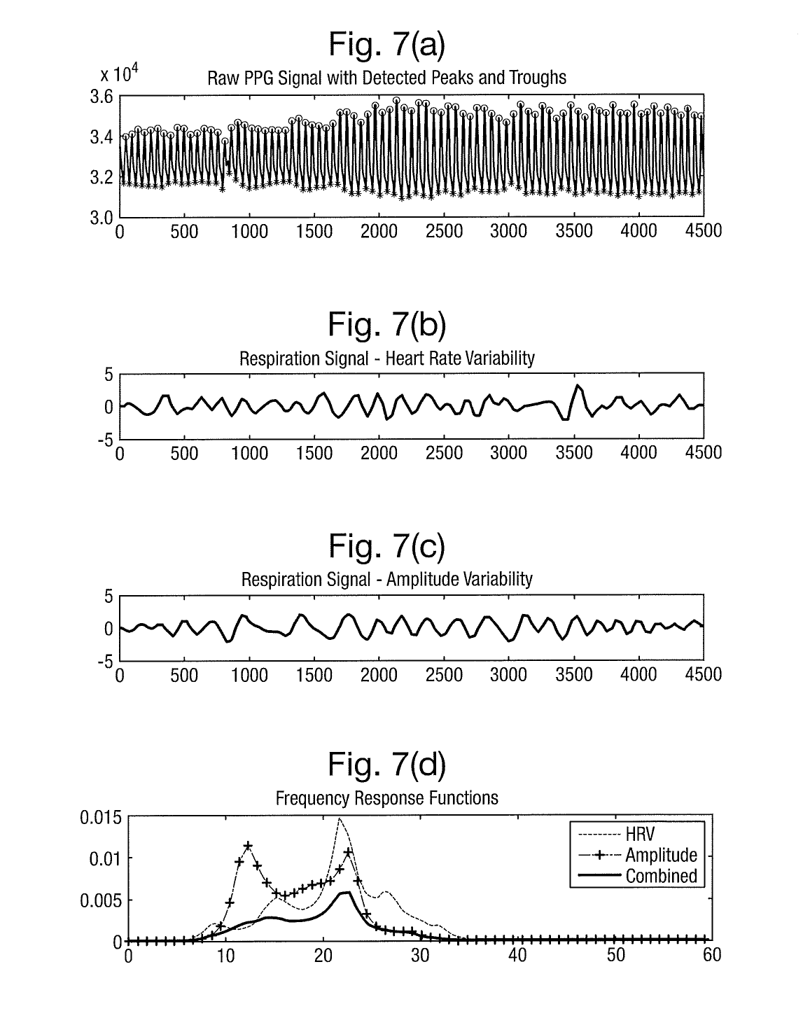

FIG. 7(a)-(d) illustrate the signal processing according to an embodiment of the present invention;

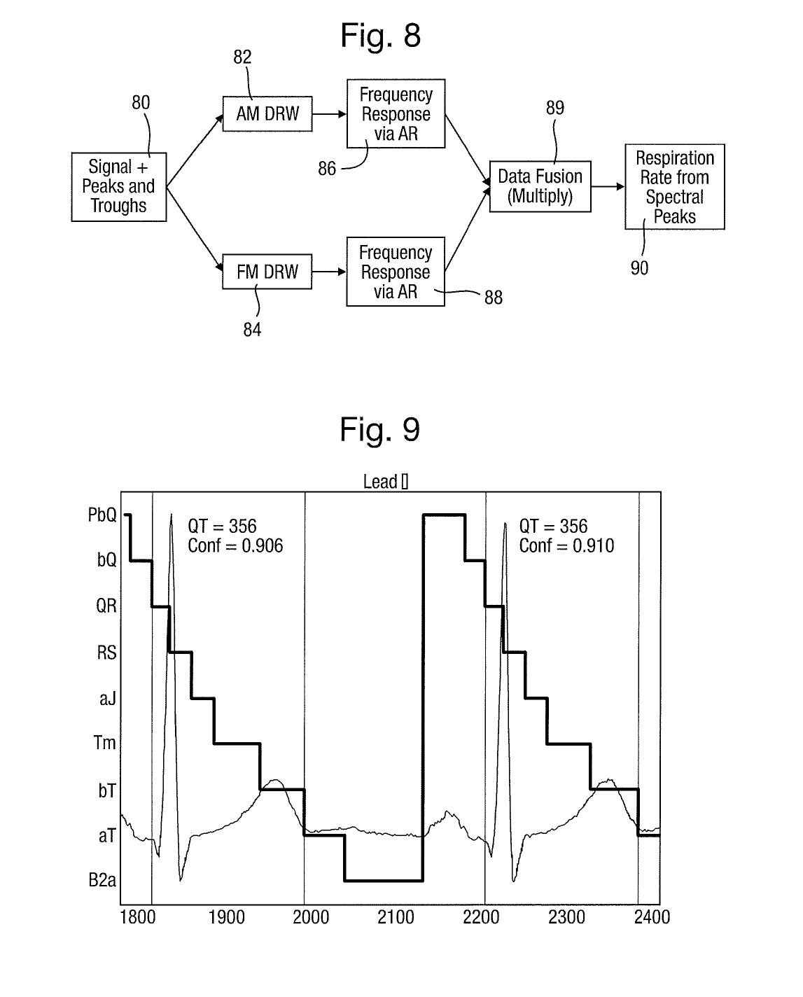

FIG. 8 illustrates schematically an embodiment of the present invention; and

FIG. 9 illustrates HMM processing of an ECG signal.

A first step of the invention is to obtain two signals representative of the respiration rate from one or more cardiac signals such as ECG or PPG signals. FIG. 1 illustrates a typical PPG signal in which each peak corresponds to one heartbeat. The peaks and troughs in the signal are located using a simple peak detection algorithm and the detected peaks and troughs are indicated in FIG. 1 by circles. The circles clearly illustrate the respiratory-based amplitude variations in the PPG waveform and in the illustration there about six breaths. Thus a derived signal representative of respiration can be obtained by performing linear or spline interpolation on the peak heights shown by the circles. Because of instantaneous changes in heart rate during respiration (respiratory sinus arrhythmia) these peaks are unevenly spaced in time. Thus another derived respiratory waveform can be obtained by measuring the peak to peak times and performing similar interpolation on the time differences. Thus two respiratory waveforms can be obtained from the PPG signal, one based on the amplitude variation and one based on the peak to peak time variation.

FIG. 2 illustrates a typical ECG waveform which can provide two further respiratory waveforms. In the case of an ECG a simple peak detection algorithm (of the type used on a PPG) cannot be used because of the presence of multiple peaks in each cycle. However the well known Pan-Tompkins algorithm can be used to identify the R-peaks and these are labelled in FIG. 2 with circles.

Incidentally, a more complex Pan-Tompkins-like algorithm could be used for peak detection in the PPG signal, and this can be beneficial in cases where a separate peak known as the dichrotic notch appears as a separate peak in the signal.

The height and location of the R-peaks in the ECG signal again provides two cardiac-derived respiratory signals, one based on the amplitude variation and one based on the peak-to-peak time variation.

An alternative way of obtaining the respiratory information from the cardiac signals is to examine the spectral content of the cardiac signals. FIG. 3 schematically illustrates the process which is to subject the signal to a Fast Fourier Transform 30, to find the fundamental frequency 32, and to zero all entries in the FFT (step 34) except for a narrow window around the fundamental, and then in step 36 to calculate the inverse FFT which results in a near sinusoidal signal at the fundamental frequency. FIG. 4 illustrates this output superimposed on the original ECG. Because the output is very smooth, the peaks and troughs can easily be detected as zero-crossings of the derivative of this signal and, if desired, the actual R- peak locations in the original ECG can be located as the maximum value of the ECG signal in a segment bracketed between two peaks or troughs of the approximate sine wave.

As an alternative to taking the fundamental in the FFT it is possible to take the approximate first harmonic of the fundamental (by zeroing all entries other than a small window around the first harmonic), and as illustrated in FIG. 5, this allows a smoother interpolation between points because there are twice as many peaks in the signal. The amplitude variation with respiration is clearly visible in FIG. 5, but a peak-to-peak time variation is also present and so, again, the two cardiac-derived respiration signals can be obtained.

FIG. 7a illustrates a PPG signal with detected peaks and troughs for a one minute (sampled at 75 Hz) measurement. FIGS. 7b and c illustrate respectively the heart rate variability and the amplitude variability represented by the identified peaks and troughs in the PPG signal. These cardiac-derived respiration signals are obtained by interpolation onto a 2 Hz time series (this frequency being chosen to allow detection of breathing rates up to the Nyquist frequency of 1 Hz or 60 breaths per minute, which is well above the normal range). The periodicity represented by breathing in the waveforms of FIGS. 7b and c is clearly visible.

In accordance with the invention these signals are then converted into the frequency domain. One way of doing this is to use the well-known Yule-Walker equations to compute AR coefficients and then the corresponding frequency response is calculated on an evenly-spaced set of frequency bins. The result of this is illustrated in FIG. 7d displayed as a thin dotted line for the heart rate variability derived signal and a thin crossed line for the amplitude derived signal. Other ways of converting the signals into the frequency domain are possible.

The two frequency response functions are then multiplied together on a point-by-point basis. This results in the thick black trace in FIG. 7d which has a single dominant peak at around 22 breaths per minute. It can be seen that this peak is close to peaks on the individual frequency response plots, but the amplitude variability trace alone shows a large, artefactual, peak at around 12 breaths per minute, which is not present in the heart rate variability derived signal. Thus the effect of multiplying the two frequency response functions together is to suppress this artefact.

FIG. 8 illustrates the process flow. In step 80 the cardiac signal (e.g. PPG or ECG) is input and the peaks and troughs corresponding to each cycle are identified. In step 82 the amplitude modulation derived signal is obtained by calculating the signal values at the times of the peaks and troughs and interpolating onto a regularly spaced interval, such as 2 Hz. In step 84 the frequency modulation derived signal is obtained by evaluating the time differences between successive peaks, again giving an unequally spaced set of intervals, which are then linearly interpolated onto the same regularly spaced interval. Both waveforms are then converted into the frequency domain in steps 86 and 88 to calculate the frequency response curves, for example by an All Poles model, and the resulting frequency response functions are then multiplied together in step 89 (by multiplying the value represented by each point in one curve by the value of the corresponding frequency point in the other curve) and the peaks in the product are analysed in step 90.

If desired a probabilistic shape detection algorithm, such as a Gaussian Mixtures model of the beat shape between peaks, can be used to identify and omit unusually shaped beats from the original raw ECG signal. Alternatively, an HMM can be used to assign a confidence value to each beat. If too many of the beats are classified as poorly formed/low confidence then the calculations are abandoned on this sample.

Having produced a product of the two frequency response curves it is necessary in step 90 to identify the peak corresponding to the breathing rate. If a single identifiable peak can be found, and the area under the curve over a narrow band either side of it (typically +/−1.5 breaths per minute) is a sufficiently high fraction of the area (typically 40-50%) of the area under the entire frequency response curve, then the frequency of that peak, converted to breaths per minute, is taken as the average breathing rate during the sample time.

It may happen that the breathing rate changes sharply during the sample time, giving rise to two or possibly three peaks in the frequency response spectrum. If this occurs, then the same area under curve calculation is performed on up to three peaks and, again, if the fraction of the total area under the combined peaks is in the region of 40-50% of the total area, then a respiration estimate is made as a weighted sum of the respiration rates corresponding to the (up to three) peaks. The weighting is determined by the relative areas under the curves of the separate peaks.

In this way the invention can provide improved estimates of the respiration rate even if the respiration rate changes up to three times during the sample period.

For continuous monitoring of respiration a rolling window of (for example) 60 seconds worth of data is maintained and the estimation procedure of the invention is made at regular intervals, for example every second - dependent on the processor power available. Furthermore, a median filter may be applied to the sequence of estimates made by the algorithm to improve the robustness of the estimation.

The invention also allows a measure of the confidence in the estimate to be obtained. The area under the combined frequency response curve calculated in a window around the estimate, as a proportion of the total area under the curve represents the strength of the identified peak. This proportion can be output as an indicator of the confidence in the estimate.

FIG. 9 illustrates schematically the HMM segmentation of an ECG in accordance with one of the known methods for such segmentation. The staircase-like trace shows the sequence of states of the HMM, which runs in a repetitive cycle going through the same sequence of states with differing durations for each beat. The embodiment of the invention described above used the peaks or troughs of the PPG or ECG signal, however by utilising the MINI additional beat-by-beat reference points are available and from these additional reference points a multiplicity of different derived waveforms representative of the heart rate and amplitude variability can be derived. Thus, for example, corresponding state transitions in each beat, from beat-to-beat, provide a time series of whole beat interval measurements and the variation in these measurements provides a heart rate variability (respiratory sinus arrhythmia) waveform. Further, the signal amplitude at any of the reference points corresponding to state transitions can be used to provide an amplitude variability signal. In addition to the beat-to-beat intervals, the variation in intra-beat intervals (for example the QT interval in the ECG) can be used to derive respiratory waveform based on respiratory sinus arrhythmia. Further, the area under the curve between two intra-beat transitions can be used to provide an amplitude variability waveform.

It will be appreciated, therefore, that a variety of respiratory indicative waveforms can be obtained from both of the PPG and ECG, and with the present invention the spectral information in these waveforms can be fused to provide an improved estimate of the respiration rate.

Although the invention has been described above with reference to methods embodying the invention, the invention can also be embodied as apparatus. For example, an apparatus for obtaining an improved estimate of respiration rate from two or more cardiac-derived signals representative of respiration, comprising:

a conversion section configured to convert the cardiac-derived signals into the frequency domain to generate respective frequency response signals representing the spectral content of the cardiac-derived signals; and

an analysis section configured to point-by-point multiply together the frequency response signals and configured to identify the improved estimate of respiration rate from one or more spectral peaks in the product function resulting from the multiplication.

Furthermore, any of the detailed method steps described above with reference to the method embodiments, such as the steps in FIG. 8, can be embodied as apparatus sections.

It is possible to implement the apparatus sections as dedicated hard-wired electronic circuits; however the various sections do not have to be separate from each other, and could all be integrated onto a single electronic chip. Furthermore, the sections can be embodied as a combination of hardware and software, and the software can be executed by any suitable general-purpose microprocessor, such that in one embodiment the apparatus can be a conventional personal computer (PC), such as a standard desktop or laptop computer, or can be a dedicated device.

The invention can also be embodied as a computer program stored on any suitable computer-readable storage medium, such as a solid-state computer memory, a hard drive, or a removable disc-shaped medium in which information is stored magnetically, optically or magneto-optically. The computer program comprises computer-executable code that when executed on a computer system causes the computer system to perform a method embodying the invention.

1. A method of obtaining an improved estimate of respiration rate from two or more cardiac-derived signals representative of respiration comprising the steps of:
converting the cardiac-derived signals into the frequency domain to generate respective frequency response signals representing the spectral content of the cardiac-derived signals;
point-by-point multiplying together the frequency response signals and identifying the improved estimate of respiration rate from one or more spectral peaks in the product function resulting from the multiplication.

2. A method according to claim 1 wherein the improved estimate of respiration rate is identified as corresponding to a single spectral peak in the product function having greater than a predetermined proportion of the total energy in the signal.

3. A method according to claim 2 wherein the proportion of total energy is determined by computing the area under the product function over a predetermined range centered on the peak and dividing it by the area under the whole function.

4. A method according to claim 1 wherein the improved estimate of respiration rate is identified as corresponding to a weighted sum of up to three peaks in the product function on condition that said up to three peaks have greater than a predetermined proportion of the total energy in the signal, where the weights are proportional to the area under the curve in a range around the peak.

5. A method according to claim 4 wherein the proportion of total energy is determined by computing the area under the product function over respective predetermined ranges centered on each of the peaks in turn, and using these as a weighted sum to compute the estimate.

6. A method according to claim 3 wherein the predetermined range corresponds to a range of respiration rates of from two to four breaths per minute centered on the respiration rate corresponding to the peak in the product function.

7. A method according to claim 2 wherein the predetermined proportion of the total energy in the signal is at least 40%.

8. A method according to claim 1 wherein the cardiac-derived signals representative of respiration comprise at least two of: a periodic amplitude variation in a photoplethysmogram or an electrocardiogram, a respiratory sinus arrhythmia in a photoplethysmogram or an electrocardiogram, a baseline variability in a photoplethysmogram.

9. A method according to claim 1 wherein the cardiac-derived signals representative of respiration are preprocessed by narrow band filtering around the expected respiration rate or a harmonic thereof.

10. A method according to any claim 1 wherein the cardiac-derived signals representative of respiration are preprocessed by use of a Hidden Markov Model to provide a beat-by-beat confidence measure and removal of sections of the signals for which the confidence measure is lower than a predetermined threshold.

11. A method according to claim 1 wherein a measure of the quality of the improved estimate of respiration rate is obtained as the proportion of the total energy represented by the one or more spectral peaks in the product function on which the improved estimate is based.

12. A method according to claim 1 wherein at least one of said cardiac-derived signals is a time series based on reference points of a cardiac signal, wherein the reference points correspond to at least one of:
a peak of the cardiac signal;
a trough of the cardiac signal; and
a state transition in a Hidden Markov Model segmentation of the cardiac signal.

13. A method according to claim 12, wherein at least one of said cardiac-derived signals is selected from:
the time series of inter-beat times from one reference point to the corresponding reference point in the next beat;
the time series of the amplitudes of the cardiac signal at corresponding reference points;
the time series of intra-beat intervals between two reference points in each beat; and
the time series of the areas under the curve between two reference points in each beat.

14. A method according to claim 12, wherein the cardiac signal is a photoplethysmogram or an electrocardiogram.

15. A computer program comprising computer-executable code that when executed on a computer system causes the computer system to perform a method according to claim 1.

16. A computer-readable medium storing a computer program according to claim 15.

17. An apparatus for obtaining an improved estimate of respiration rate from two or more cardiac-derived signals representative of respiration, comprising:
a conversion section configured to convert the cardiac-derived signals into the frequency domain to generate respective frequency response signals representing the spectral content of the cardiac-derived signals; and
an analysis section configured to point-by-point multiply together the frequency response signals and configured to identify the improved estimate of respiration rate from one or more spectral peaks in the product function resulting from the multiplication.
